(19) United States
(12) Patent Application Publication
(10) Pub. No.: US 2021/0244527 A1
(43) Pub. Date: Aug. 12, 2021

(54) Silicone Prosthesis Delivery Apparatus and Methods of Use

(71) Applicant: Conical Cover LLC, Boca Raton, FL (US)

(72) Inventor: Scott Hyler Heneveld, Whitmore, CA (US)

(73) Assignee: Conical Cover LLC, Boca Raton, FL (US)

(21) Appl. No.: 17/163,941

(22) Filed: Feb. 1, 2021

Related U.S. Application Data
(60) Provisional application No. 62/971,993, filed on Feb. 9, 2020.

(51) Int. Cl. A61F 2/12

(52) U.S. Cl. CPC A61F 2/12; A61F 2230/0067; A61F 2230/001

(57) ABSTRACT

A silicone prosthesis delivery apparatus and associated methods are disclosed for facilitating the insertion of a silicone prosthesis into a surgically developed pocket of a patient. In at least one embodiment, a flexible, substantially hourglass-shaped delivery sleeve is configured for receiving and subsequently expelling the prosthesis therefrom. The delivery sleeve provides a substantially conical-shaped transfer channel and a substantially conical-shaped delivery chamber, the transfer channel and delivery chamber opposingly positioned and joined with one another so as to form a relatively smaller diameter passage therebetween. A free end of the transfer channel provides an entry opening configured for allowing the prosthesis to selectively pass therethrough when the prosthesis is inserted into the delivery sleeve, and a free end of the delivery chamber provides a closed end.

### RELATED APPLICATIONS

This application claims priority and is entitled to the filing date of U.S. provisional application Ser. No. 62/971,993, filed on Feb. 9, 2020. The contents of the aforementioned application are incorporated herein by reference.

### BACKGROUND

The subject of this patent application relates generally to prothesis delivery devices, and more particularly to a silicone prosthesis delivery apparatus and associated methods of use for facilitating the insertion of a silicone implant, such as a breast prosthesis, into a surgically developed pocket of a patient.

Applicant hereby incorporates herein by reference any and all patents and published patent applications cited or referred to in this application.

By way of background, silicone implants have been in worldwide use for many years. While marketing of such implants was halted within the United States for a period of time, the use of silicone implants has resumed within the United States. Traditional surgical approaches of inserting prefilled silicone implants require the use of larger incisions in comparison to saline implants which can be inserted through small incisions which are later filled in situ with saline. While many patients prefer the more natural qualities of silicone, patients remain apprehensive because of the larger incisions and possibility for visible scars which can result from silicone implants.

An additional concern with the use of silicone implants is that the longevity and integrity of the implants can be damaged by the conventional insertion process. A typical insertion process involves direct finger handling and prodding by the surgeon of the implant in order to insert it into the surgical pocket. Implant failures can be associated with an area of high stress to the outer surface of the implant. The stressed areas are believed to correlate to excessive pressure applied by finger handling and prodding of the implant and/or damage associated with damage of the implant surface by a “touching” injury that damages the implant.

Another concern is contamination of the implant upon placement in the breast pocket. Capsular contracture is the leading complication after breast augmentation. The contracture is believed to be the result of a low-grade bacterial infection or the formation of a biofilm around implants that causes severe inflammation. Although bacterial contamination has been implicated in capsule formation, the process of contracture is thought to be multifactorial, including inflammatory responses of the immune system. There are several factors that contribute to capsular contracture: 1) implant texture, 2) implant type, 3) incision type, 4) rupture/leakage, and 5) pocket contamination with blood, bacteria, and synthetic fibers. Many of these factors can be controlled, for example by using antimicrobial implant baths and pocket irrigation, along with refining surgical techniques to minimize the implant's contact with the surgeon's gloves and patient's skin. The use of triple-antibiotic breast pocket irrigation has been proven to greatly reduce the incidence of capsular contracture in breast augmentations. The development of the Keller Funnel, a mechanical insertion device, allowed for a no-touch implant technique by giving an alternative to hand-placement of implants into breast pockets. The Keller Funnel is constructed of vinyl film with a lubricous hydrophilic coating. The exit opening of the Keller Funnel is cut to the implant size and then hydrated before the implant is poured directly from the packaging into the funnel. The funnel is placed about 1 centimeter inside the dissected pocket, and the implant is expelled through the funnel and into the pocket as a no-skin touch technique. The funnel makes implant insertion safer by decreasing the stress to the implant shell, minimizing contact with the patient's skin and the contact with the surgeon's gloves during insertion. The funnel also affords time savings by reducing surgical duration for implant insertion.

However, due to the expensive lubricious hydrophilic coating utilized by the Keller Funnel, much of the cost savings attributed to reduced surgical time and improved outcomes are negated. Although the clinical benefits are documented and appreciated by plastic surgeons, the high cost of the device prevents many surgeons from adopting the device in their practice.

In addition, the high cost prohibits the use of a separate funnel for each the left and right breasts. The same funnel is used for both sides, thus inducing the risk of cross-contamination on the second implantation. Multiple uses of the device can result in sloughing of the surface hydrophilic coating, thus exposing the high friction vinyl substrate to the silicone implant shell. This could result in elevated stress on the implant shell during implantation, with possible damage or susceptibility to bacterial infection or biofilm formation. The Keller Funnel also has a large opening to allow entry of the silicone implant. This large opening provides a means for the silicone implant to unintentionally slide from the funnel and off the sterile field, thus requiring vigilance by the clinician to maintain the implant inside the funnel.

An additional consideration with respect to silicone implants involves the amount of time required to insert the implants. A traditional hand manipulation of an implant into a surgical pocket can take between 10 to 20 minutes per implant for even a highly skilled surgical practitioner. Typically, hand manipulation of an implant requires the use of a larger incision and would be done with a saline implant. The amount of time required has a direct bearing on the expense of the procedure, the surgical expense reflecting the surgeon's time, the support staff within the operating room, and the amount of time allocated for the surgical procedure. Accordingly, any improvements to reduce the time required for implantation of the silicone implant will have significant cost savings with respect to the surgical procedure.

Thus, there remains a need for a cost-effective funnel for delivering breast implants that will allow more patients to clinically benefit from a no-skin touch technique, while also reducing the attention needed by the clinician to prevent the implant from unintentionally escaping from the funnel. Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a silicone prosthesis delivery apparatus and associated methods for facilitating the insertion of a silicone prosthesis into a surgically developed pocket of a patient. In at least one embodiment, a flexible, substantially hourglass-shaped delivery sleeve is configured for receiving and subsequently expelling the prosthesis therefrom. The delivery sleeve provides a substantially conical-shaped transfer channel and a substantially conical-shaped delivery chamber, the transfer channel and delivery chamber opposingly positioned and joined with one another so as to form a relatively smaller diameter passage therebetween. A free end of the transfer channel provides an entry opening configured for allowing the prosthesis to selectively pass therethrough when the prosthesis is inserted into the delivery sleeve, and a free end of the delivery chamber provides a closed end. With the prosthesis positioned within the transfer channel, the transfer channel is capable of being manipulated to apply pressure to direct the prosthesis along a length of the delivery sleeve and toward the delivery chamber, such that the prosthesis may be urged through the passage and into the delivery chamber. With the prosthesis subsequently positioned within the delivery chamber, the delivery chamber is configured for being selectively separated from the transfer channel at a location proximal the passage, thereby forming a trimmed opening at a proximal end of the delivery chamber, opposite the closed end, the trimmed opening sized and configured for allowing the prosthesis to selectively pass therethrough when the prosthesis is expelled from the delivery chamber. The delivery chamber is then capable of being manipulated to apply pressure to direct the prosthesis along a length of the delivery sleeve and toward the trimmed opening, such that the prosthesis may be expelled from the delivery chamber through the trimmed opening.

An exemplary method for preparing the silicone prosthesis delivery apparatus includes the steps of inserting a prosthesis through the entry opening of the transfer channel of the delivery sleeve, such that the prosthesis is positioned within the transfer channel; manipulating the transfer channel to apply pressure to direct the prosthesis along a length of the delivery sleeve and toward the delivery chamber, thereby urging the prosthesis through the passage and into the delivery chamber; separating the delivery chamber from the transfer channel, thereby forming the trimmed opening sized for preventing the prosthesis from unintentionally exiting the delivery chamber; and positioning the delivery sleeve within a sterile barrier packaging.

It is one aspect of at least one of the present embodiments to provide an apparatus and a process for facilitating the delivery of a silicone implant into a surgically developed pocket of a patient.

It is a further aspect of at least one embodiment of the present invention to provide for an apparatus and process for facilitating the placement of a filled silicone implant into a surgical pocket through a surgical incision that is too small for a manual insertion of an implant.

It is a further aspect of at least one of the present embodiments to provide for an apparatus and process that allows insertion of a silicone implant through a sleeve defining a small diameter outlet into a patient without direct hand manipulation of the implant.

It is yet a further and more particular aspect of at least one aspect of at least one of the present embodiments to provide for a process and apparatus that allows for the retention of a silicone implant in a sleeve for a “touchless” insertion of a silicone implant into a surgical pocket. The construct allows for the placement of the implant within the delivery chamber for subsequent retention of the implant within the apparatus.

It is yet a further and more particular aspect of at least one aspect of at least one of the present embodiments to provide for a process and apparatus that allows for the silicone implant to be “pre-loaded” at the factory into the sleeve and then placed together as an assembly in a sterile barrier package for sterilization and then delivered to the hospital setting to provide the facilitation of a truly “touchless” insertion of a silicone implant into a surgical pocket.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a perspective view of an exemplary silicone prosthesis delivery apparatus along with an exemplary silicone prosthesis, in accordance with at least one embodiment;

FIG. 2 is a further perspective view thereof, illustrating the prosthesis positioned within a delivery chamber of the apparatus, in accordance with at least one embodiment;

FIG. 3 is a perspective view of a further exemplary silicone prosthesis delivery apparatus along with an exemplary silicone prosthesis, in accordance with at least one embodiment;

FIG. 4a is a perspective view thereof, illustrating the prosthesis positioned within the delivery chamber of the apparatus, in accordance with at least one embodiment;

FIG. 4b is a perspective view thereof, illustrating the prosthesis positioned within the delivery chamber of the apparatus, and the delivery chamber positioned within a structural sleeve of the apparatus, in accordance with at least one embodiment;

FIG. 5 is a perspective view of a still further exemplary silicone prosthesis delivery apparatus along with an exemplary silicone prosthesis, in accordance with at least one embodiment;

FIG. 6 is a perspective view of a still further exemplary silicone prosthesis delivery apparatus, in accordance with at least one embodiment;

FIG. 7 is a perspective view of an exemplary stretching tool, in accordance with at least one embodiment;

FIG. 8 is a perspective view of an exemplary silicone prosthesis delivery apparatus along with an exemplary silicone prosthesis, illustrating the prosthesis positioned within the delivery chamber of the apparatus, along with the apparatus being positionable within a transport container, in accordance with at least one embodiment;

FIG. 9 is a diagram illustrating a prior art method for utilizing a prior art delivery funnel to insert a silicone implant into a surgically developed pocket of a patient; and

FIG. 10 is a diagram illustrating an exemplary method of utilizing the exemplary silicone prosthesis delivery apparatus to insert a silicone implant into a surgically developed pocket of a patient, in accordance with at least one embodiment.

The above-described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

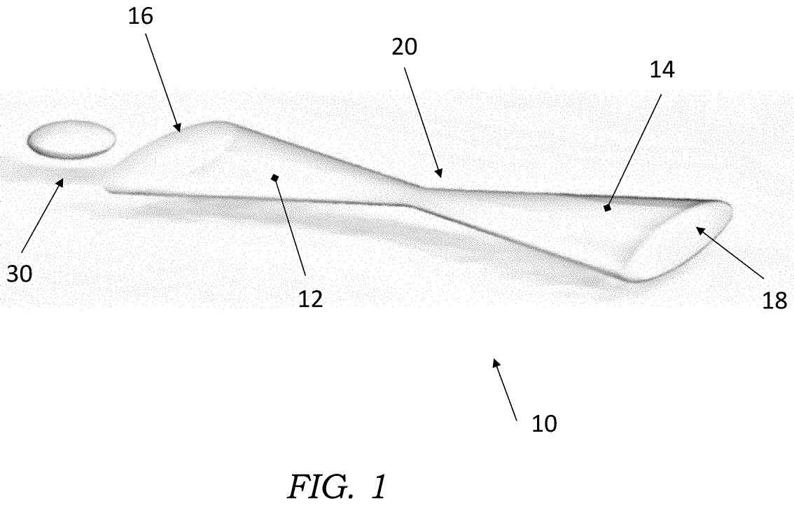

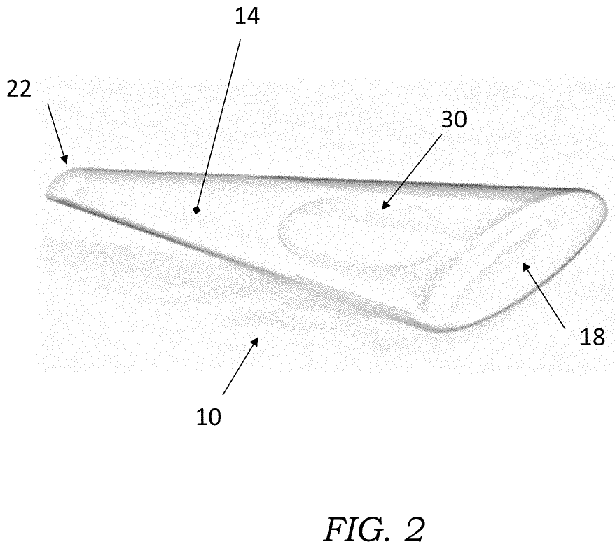

Turning now to FIGS. 1 and 2, there are shown perspective views of an exemplary embodiment of a silicone prosthesis delivery apparatus configured for facilitating the insertion of an at least one silicone prosthesis 30 into a surgically developed pocket of a patient (hereinafter referred to generally as a “patient pocket” for simplicity purposes), such as a breast pocket for example. In at least one embodiment, the apparatus comprises a substantially hourglass-shaped delivery sleeve 10 having two opposingly joined, generally conical-shaped portions (hereinafter referred to as a delivery chamber 14 and a transfer channel 12, respectively) which cooperate to define a entry opening 16 at one end of the delivery sleeve 10 (i.e., at a free end of the transfer channel 12), a closed end 18 at the opposing end of the delivery sleeve 10 (i.e., at a free end of the delivery chamber 14), and a relatively smaller passage 20 positioned therebetween (i.e., where the transfer channel 12 is joined with the delivery chamber 14). In at least one embodiment, the transfer channel 12 and delivery chamber 14 have similar shapes, dimensions and materials of construction. However, in at least one alternate embodiment, the transfer channel 12 and delivery chamber 14 may be of differing shapes, sizes and/or materials. In at least one embodiment, each of the delivery chamber 14 and transfer channel 12 are constructed out of a material which is sufficiently flexible, thereby enabling the delivery sleeve 10 to assume a flattened configuration for storage and shipping when not in use.

With continued reference to FIGS. 1 and 2, in at least one embodiment, when the prosthesis 30, such as a silicone implant, is placed through the entry opening 16 into the transfer channel 12, the transfer channel 12 can be manipulated to conform to the shape of the prosthesis 30 as well as to apply pressure to direct the prosthesis 30 along the length of the delivery sleeve 10 and toward the delivery chamber 14. Adequate manipulation can traverse the prosthesis 30 though the passage 20 and into the delivery chamber 14. In at least one embodiment, the delivery sleeve 10 may be cut at a location proximal the passage 20 to create a trimmed opening 22 at a proximal end of the delivery chamber 14, opposite the closed end 18 of the delivery chamber 14. In at least one such embodiment, the trimmed opening 22 is of a defined size to allow the extrusion and subsequent delivery of the prosthesis 30 into the patient pocket. In at least one embodiment, an outer surface of the delivery sleeve 10 provides markings or other indicia positioned and configured for aiding in locating the desired position for cutting the delivery sleeve 10 to form the trimmed opening 22. In at least one alternate embodiment, the transfer channel 12 and delivery chamber 14 are removably engaged with one another, such that disengaging the delivery chamber 14 from the transfer channel 12 automatically forms the trimmed opening 22. In at least one embodiment, the delivery chamber 14 can be manipulated to conform to the shape of the prosthesis 30 as well as to apply pressure to direct the prosthesis 30 along the length of the delivery chamber 14 and extrude the prosthesis 30 through the trimmed opening 22.

In at least one embodiment, both the delivery chamber 14 and transfer channel 12 are constructed of flexible materials. The materials could be compatible for joining the delivery chamber 14 and transfer channel 12 by means of heat seal bonding, RF welding, adhesive bonding, or other appropriate means—now known or later developed. In at least one alternate embodiment, the delivery chamber 14 and transfer channel 12 are of a single, unitary construction. In at least one embodiment, the delivery chamber 14 is constructed out of a flexible material. In at least one such embodiment, the delivery chamber 14 is constructed out of a fabric material, such as a plastic-containing fabric which is pliable yet resistant to stretching. In at least one further embodiment, the delivery chamber 14 is made of a latex rubber or other material with similar elastic properties—now known or later developed—to enable low force expansion of the trimmed opening 22. By way of non-limiting example, the elastomer may be in the durometer range of 20-90 Shore A with a percent elongation in the range of 300-800 percent. The material may be thin and, or, have slits or holes in the geometry to further enhance low force expansion. It is also envisioned that use of a transparent plastic or other suitable polymer material which has sufficient properties including flexibility and non-elasticity may be used. It is believed that there are advantages to using a transparent or semitransparent material to assist the surgeon in proper orientation of the prosthesis 30 within the delivery sleeve 10. Suitable transparent materials may include mylar, vinyl, LDPE, polyurethane, polyvinyl, chloride and other similar materials—now known or later developed. As disclosed therein, suitable films heat sealed to form suitable containers, are transparent with minimal hazing, and can be sterilized using gas sterilization, irradiation sterilization, or heat with intact seals and remain sufficiently flexible and pliable for the necessary handling described herein. In at least one embodiment, the transfer channel 12 is flexible and may be elastic, made of a latex or other material with similar elastic properties—now known or later developed—to enable low force expansion of the entry opening 16. The material may be thin and/or have slits or holes in the geometry to further enhance low force expansion.

In at least one embodiment, other attributes of the delivery sleeve 10 include the ability for the delivery sleeve 10 to be a sterile component. Accordingly, in such embodiments, the materials must be capable of withstanding at least one of several conventional sterilization techniques such as a steam autoclave, chemical gas sterilization, or irradiation for example. Additionally, in at least one embodiment, an interior surface of the delivery sleeve 10 provides a low coefficient of friction to facilitate passage of the prosthesis 30 through the delivery sleeve 10. In at least one embodiment, the delivery sleeve 10 is constructed of a film material with a lubricious additive dispersed within the film during the processing of the film sheet. Slip is an additive that could be utilized in the film in at least one embodiment. Slip agents have a part that is soluble in the substrate and a part that is insoluble. During processing (in the molten phase), slip additives (as overall effect) are soluble and homogeneously dispersed in the substrate. Upon crystallization, the solubility limit is exceeded and the slip additive migrates from the matrix toward the surface, thus significantly reducing the coefficient of friction at the film surface. Fatty acid amides, including primary and secondary amides, are commonly used slip additives. Anti-blocking additives may also be utilized in at least one embodiment. Anti-blocking helps prevent adjacent film layers from sticking to each other due to electrostatic charge or Van der Waals forces (attractive forces between polymer chains). Incorporating anti-blocking properties creates a micro-rough surface that reduces adhesion between adjacent film layers and helps prevent damage. Alternatively, in at least one embodiment, wax additives could be dispersed in the base material, such as polyurethane, to modify the surface appearance, feel, slip, abrasion resistance and overall durability of the film. The expense of additives integrated during the film manufacturing process is relatively low, especially compared to hydrophilic coatings applied to finished film. In at least one embodiment, the film could also be processed to have a textured surface to enhance lubricity. In still further embodiments, any other lubricant (or any other material or composition having sufficient lubricious properties), now known or later developed, may be substituted. Such lubricants may be applied directly to the prosthesis 30 or the delivery sleeve 10 can be coated with lubricant or supplied pre-coated with a lubricant that is already present within the delivery sleeve 10.

Since the size of the prosthesis 30 may vary in a range from about 150 cc to approximately 800 cc, the dimensions of each of the entry opening 16 and trimmed opening 22 may vary in order to accommodate various sizes for the prosthesis 30. In at least one embodiment, the passage 20 is sized for accommodating the smallest-sized prosthesis 30. Additionally, in at least one embodiment, one or both of the entry opening 16 and trimmed opening 22 may be selectively enlarged by cutting portions of the delivery sleeve 10, proximal the entry opening 16 or trimmed opening 22, to provide for larger openings. In at least one embodiment, the outer surface of the delivery sleeve 10 provides markings or other indicia positioned and configured for assisting with the cutting of the delivery sleeve 10 to the appropriate dimensions for the size of the prosthesis 30.

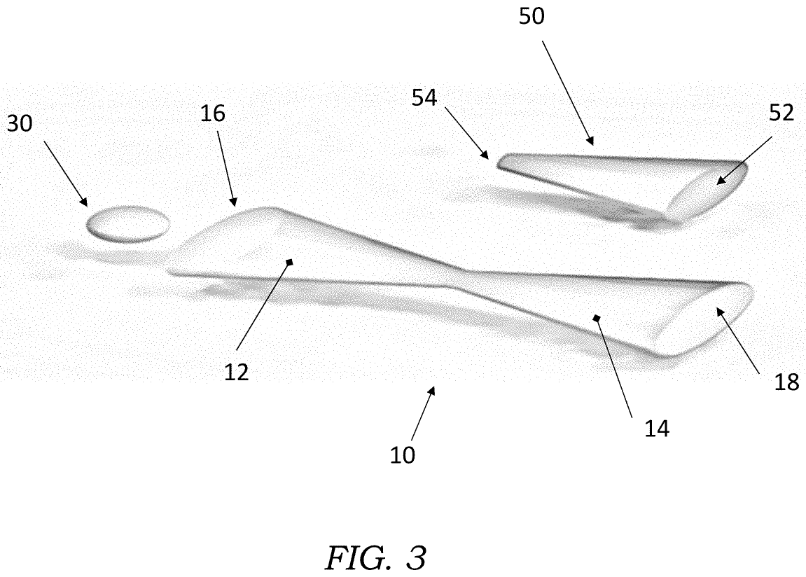

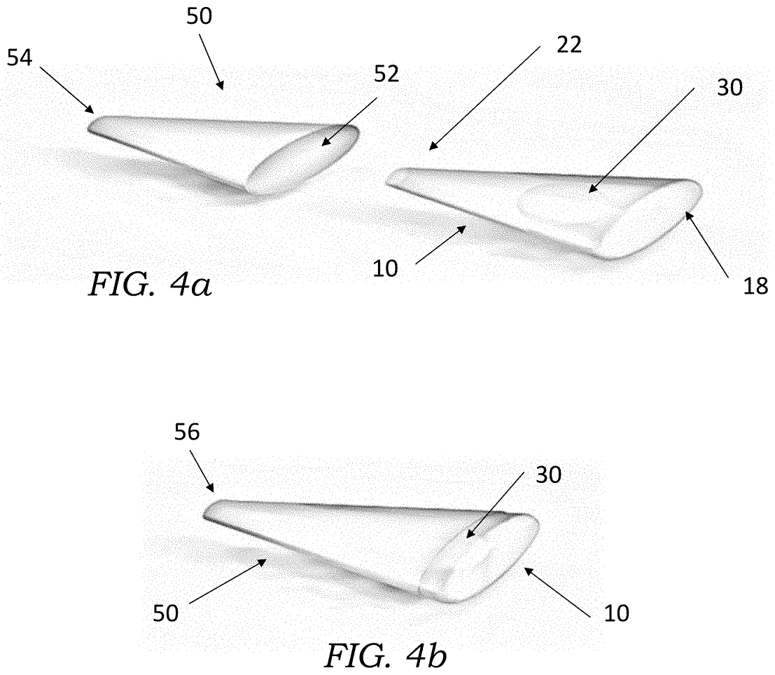

In at least one further embodiment, as illustrated in FIGS. 3, 4a and 4b, the apparatus further provides a substantially conical-shaped structural sleeve 50 having a first opening 52 and an opposing, relatively smaller second opening 54. In at least one embodiment, the taper of the structural sleeve 50 may be of similar geometry to that of the delivery chamber 14 of the delivery sleeve 10. In at least one embodiment, as illustrated in FIG. 4b, the structural sleeve 50 is sized and configured for removably receiving the delivery chamber 14 therewithin. Accordingly, in at least one such embodiment, the structural sleeve 50 is sized for approximating the dimensions of the delivery chamber 14. When fitted together, the delivery chamber 14 and structural sleeve 50 may be cut at a location proximal the second opening 54 to create a uniform trimmed opening 56. In at least one embodiment, the uniform trimmed opening 56 is of a defined size to allow the extrusion and subsequent delivery of the prosthesis 30 into the patient pocket. In at least one embodiment, an outer surface of the structural sleeve 50 provides markings or other indicia positioned and configured for assisting with the cutting of the structural sleeve 50 to the appropriate dimensions for the size of the prosthesis 30. Alternatively, the structural sleeve 50 may be trimmed near the second end 54 prior to inserting the delivery chamber 14 into the structural sleeve 50. In at least one embodiment, the trimmed opening 22 of the delivery chamber 14 could be a different size than the second end 54 of the structural sleeve 50. After fitted together, the structural sleeve 50 and delivery chamber 14 can be manipulated together to conform to the shape of the prosthesis 30 as well as to apply pressure to direct the prosthesis 30 along the length of the delivery chamber 14 and extrude the prosthesis 30 through the uniform trimmed opening 56.

In at least one embodiment, the structural sleeve 50 is constructed out of a flexible and relatively inelastic material, such as vinyl for example. In at least one alternate embodiment, the structural sleeve 50 is constructed out of a fabric material, such as a plastic-containing fabric for example, which is pliable yet resistant to stretching. It is also envisioned that use of a transparent plastic or other suitable polymer material which has sufficient properties including flexibility and non-elasticity may be used. It is believed that there are advantages to using a transparent or semitransparent material to assist the surgeon in proper orientation of the prosthesis 30 within the delivery sleeve 10. Suitable transparent materials may include mylar, vinyl, LDPE, polyurethane, polyvinyl chloride, silicone, and other similar materials—now known or later developed. In at least one such embodiment, the structural sleeve 50 is constructed out of compositions of ethylene and alpha-olefin copolymers, such as the compositions used in IV saline bags, which are both flexible and transparent. In at least one such embodiment, the structural sleeve 50 is constructed out of heat resistant silicone suitable for autoclave sterilization for subsequent reuse. As disclosed therein, suitable films heat sealed to form suitable containers, are transparent with minimal hazing, and can be sterilized using gas sterilization, irradiation sterilization, or heat with intact seals and remain sufficiently flexible and pliable for the necessary handling described herein.

In at least one embodiment, other attributes of the structural sleeve 50 include the ability for the structural sleeve 50 to be a sterile component, similar to the delivery sleeve 10. Accordingly, in such embodiments, the materials must be capable of withstanding at least one of several conventional sterilization techniques such as a steam autoclave, chemical gas sterilization, or irradiation for example. Additionally, in at least one embodiment, an interior surface of the structural sleeve 50 (or, alternatively, an outer surface of the delivery chamber 14) provides a low coefficient of friction to facilitate insertion of the delivery chamber 14 into the structural sleeve 50 through the first end 52. In at least one embodiment, the structural sleeve 50 provides a film material with a lubricious additive dispersed within the film during the processing of the film sheet. As discussed above, slip is an additive that could be utilized in the film in at least one embodiment. Anti-blocking additives may also be utilized in at least one embodiment. Alternatively, in at least one embodiment, wax additives could be dispersed in the base material, such as polyurethane, to modify the surface appearance, feel, slip, abrasion resistance and overall durability of the film. In at least one embodiment, the film could also be processed to have a textured surface to enhance lubricity. In still further embodiments, any other lubricant (or any other material or composition having sufficient lubricious properties), now known or later developed, may be substituted. The structural sleeve 50 and/or delivery chamber 14 can be coated with lubricant or supplied pre-coated with a lubricant that is already present thereon.

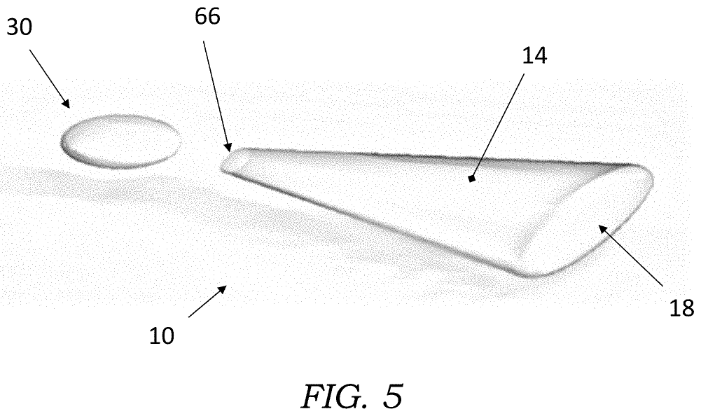

In at least one further embodiment, as illustrated in FIG. 5, the transfer channel 12 may be omitted, such that the delivery sleeve 10 simply provides the delivery chamber 14. In such embodiments, the delivery chamber 14 provides a chamber opening 66 opposite the closed end 18, with the chamber opening 66 configured for being selectively stretched to accommodate passage of the prosthesis 30 into the delivery chamber 14 and then released to retain the prosthesis 30 within the delivery chamber 14. The construct could be similar to those embodiments noted above.

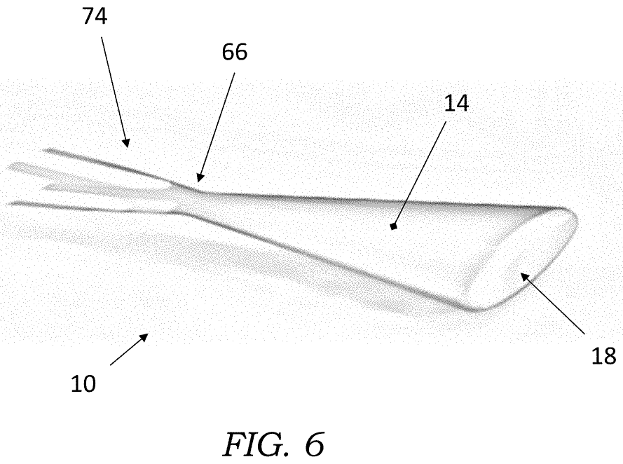

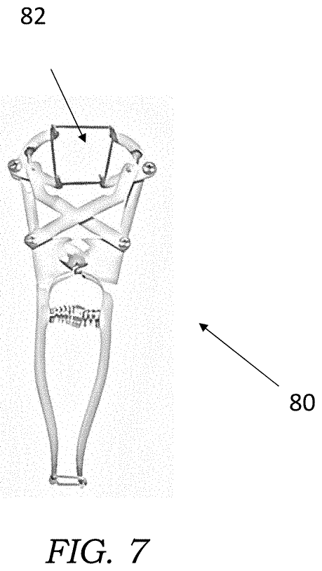

In at least one still further embodiment, as illustrated in FIG. 6, the chamber opening 66 provides a plurality of tabs 74 extending outwardly therefrom, the tabs 74 being positioned and configured for facilitating the selective stretching of the chamber opening 66 to an adequate size for allowing the prosthesis 30 to pass therethrough, into the delivery chamber 14, and then subsequently released to reduce the size of the chamber opening 66. The delivery chamber 14 may then be trimmed near the chamber opening 66 to remove the tabs 74 and create a trimmed opening. FIG. 7 illustrates a stretching tool 80 that may optionally be used to perform the function of stretching the chamber opening 66. The tool 80 may expand and maintain the chamber opening 66 in the enlarged state while the prosthesis 30 is delivered into the delivery chamber 14. The tool 80 may then be contracted to release tension on the chamber opening 66 and then the tool 80 may be released from the delivery sleeve 10. Other configurations or variations of such an expanding tool—now known or later developed—may also be used in at least one embodiment, along with combinations of such expanding tools working in conjunction with tab 74 features on the delivery chamber 14. Thereafter, the surgeon is able to carry or manipulate the delivery chamber 14 without the prosthesis 30 unintentionally escaping and falling out of the delivery chamber 14. The surgeon is thus able to apply pressure to the prosthesis 30, forcing the prosthesis 30 toward the chamber opening 66.

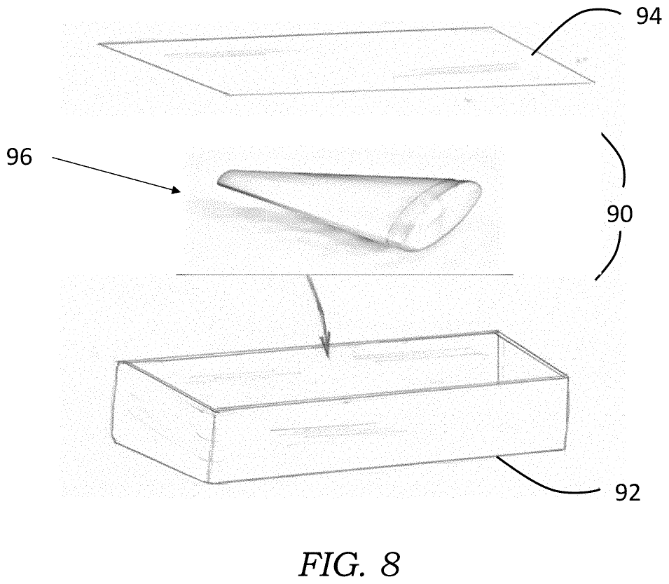

Another aspect of the invention involves an exemplary method of placing the prosthesis 30 within the delivery sleeve 10, and placing the assembly 96 within the appropriate sterile barrier packaging 90 (FIG. 8) prior to sterilization and shipment to the customer. In at least one embodiment, the sterile barrier packaging 90 could be a similar tray 92 and lid 94 commonly used for sterilization and shipment of implants. In at least one embodiment, the tray 92 could contain saline solution or other fluids to hydrate the prosthesis 30 and delivery sleeve 10. In at least one embodiment, the exemplary method would eliminate the concern of contamination in the operating room when the prosthesis 30 is being transferred and loaded into the delivery sleeve 10. In at least one embodiment, the passage 20 (or trimmed opening 22) would aid in containing and maintaining the prosthesis 30 within the delivery sleeve 10 during transport. In at least one embodiment, one or more of structural sleeve 50 could be provided in a separate package. Due to the structural sleeve 50 not requiring an expensive lubricious coating, the cost reduction could provide the economic advantage of each delivery sleeve 10 using a fresh, sterile structural sleeve 50, thus the potential to mitigate cross-contamination of the patient pockets.

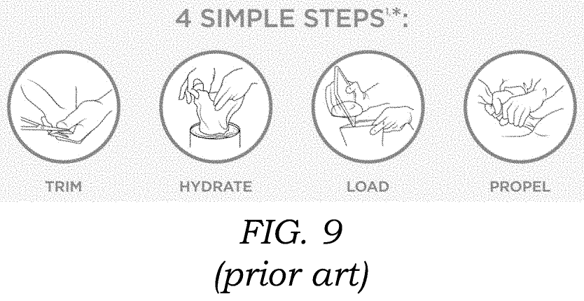

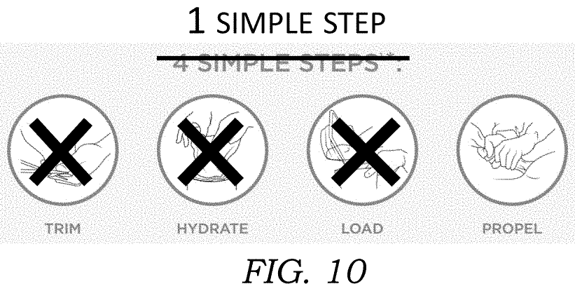

FIG. 9 provides a diagram that illustrates the known prior art funnel delivery method and handling of the prosthesis 30. Four steps are involved, three of which can expose the prosthesis 30 to contamination. By comparison, FIG. 10 provides a diagram that illustrates the exemplary method of utilizing the apparatus, where the prosthesis 30 is placed within the delivery sleeve 10 prior to being placed in the sterile barrier packaging 90. The three steps which can expose the prosthesis 30 to contamination are eliminated, thus providing improved sterile integrity and cleanliness to the prosthesis 30.

Aspects of the present specification may also be described as the following embodiments:

1. A silicone prosthesis delivery apparatus for facilitating the insertion of a silicone prosthesis into a surgically developed pocket of a patient, the apparatus comprising: a substantially hourglass-shaped delivery sleeve configured for receiving and subsequently expelling the prosthesis therefrom, the delivery sleeve comprising: a substantially conical-shaped transfer channel and a substantially conical-shaped delivery chamber, the transfer channel and delivery chamber opposingly positioned and joined with one another so as to form a relatively smaller diameter passage therebetween; a free end of the transfer channel providing an entry opening configured for allowing the prosthesis to selectively pass therethrough when the prosthesis is inserted into the delivery sleeve; and a free end of the delivery chamber providing a closed end; whereby, with the prosthesis positioned within the transfer channel, the transfer channel is capable of being manipulated to apply pressure to direct the prosthesis along a length of the delivery sleeve and toward the delivery chamber, such that the prosthesis may be urged through the passage and into the delivery chamber; whereby, with the prosthesis subsequently positioned within the delivery chamber, the delivery chamber is configured for being selectively separated from the transfer channel at a location proximal the passage, thereby forming a trimmed opening at a proximal end of the delivery chamber, opposite the closed end, the trimmed opening sized and configured for allowing the prosthesis to selectively pass therethrough when the prosthesis is expelled from the delivery chamber; and whereby, with the prosthesis positioned within the delivery chamber, the delivery chamber is capable of being manipulated to apply pressure to direct the prosthesis along a length of the delivery sleeve and toward the trimmed opening, such that the prosthesis may be expelled from the delivery chamber through the trimmed opening.

2. The silicone prosthesis delivery apparatus according to embodiment 1, wherein the delivery sleeve is constructed out of a flexible material.

3. The silicone prosthesis delivery apparatus according to embodiments 1-2, wherein one or both of the transfer channel and deliver chamber are constructed out of a flexible material having elastic properties.

4. The silicone prosthesis delivery apparatus according to embodiments 1-3, wherein the delivery sleeve is constructed out of a film material having a lubricious additive dispersed therewithin.

5. The silicone prosthesis delivery apparatus according to embodiments 1-4, wherein the lubricious additive is a slip agent.

6. The silicone prosthesis delivery apparatus according to embodiments 1-5, wherein the delivery sleeve further provides an anti-blocking additive.

7. The silicone prosthesis delivery apparatus according to embodiments 1-6, wherein the delivery sleeve further provides a wax additive.

8. The silicone prosthesis delivery apparatus according to embodiments 1-7, wherein the delivery sleeve provides a textured inner surface for enhancing lubricity of the delivery sleeve.

9. The silicone prosthesis delivery apparatus according to embodiments 1-8, wherein an inner surface of the delivery sleeve provides a lubricant.

10. The silicone prosthesis delivery apparatus according to embodiments 1-9, wherein an inner surface of the delivery sleeve provides a hydrophilic coating.

11. The silicone prosthesis delivery apparatus according to embodiments 1-10, wherein the delivery sleeve is transparent or semi-transparent.

12. The silicone prosthesis delivery apparatus according to embodiments 1-11, wherein the diameter of the passage is sized for preventing the prosthesis from unintentionally exiting the delivery chamber.

13. The silicone prosthesis delivery apparatus according to embodiments 1-12, wherein the delivery sleeve is capable of being cut proximal one or both of the entry opening and trimmed opening, thereby allowing one or both of the entry opening and trimmed opening to be selectively enlarged to better accommodate the prosthesis as needed.

14. The silicone prosthesis delivery apparatus according to embodiments 1-13, wherein an outer surface of the delivery sleeve provides markings or other indicia positioned and configured for assisting with the cutting of the delivery sleeve to selectively enlarge one or both of the entry opening and trimmed opening.

15. The silicone prosthesis delivery apparatus according to embodiments 1-14, wherein the delivery chamber is configured for being selectively cut at a location proximal the passage, thereby separating the delivery chamber from the transfer channel.

16. The silicone prosthesis delivery apparatus according to embodiments 1-15, wherein an outer surface of the delivery sleeve provides markings or other indicia positioned and configured for assisting with the cutting of the delivery sleeve to form the trimmed opening.

17. The silicone prosthesis delivery apparatus according to embodiments 1-16, wherein the delivery chamber and transfer channel are removably engaged with one another at a location proximal the passage, such that disengaging the delivery chamber from the transfer channel automatically forms the trimmed opening.

18. The silicone prosthesis delivery apparatus according to embodiments 1-17, further comprising: a substantially conical-shaped structural sleeve sized and configured for removably receiving the delivery chamber therewithin after the delivery chamber has been separated from transfer channel, thereby providing structural integrity to the delivery chamber; the structural sleeve having a first opening sized for receiving the delivery chamber therethrough, and an opposing, relatively smaller second opening sized for accommodating the trimmed opening of the inserted delivery chamber; whereby, with the delivery chamber positioned within the structural sleeve, the delivery chamber and structural sleeve are configured for being simultaneously cut at a location proximal the second opening, thereby forming a uniform trimmed opening sized and configured for allowing the prosthesis to selectively pass therethrough when the prosthesis is expelled from the delivery chamber.

19. The silicone prosthesis delivery apparatus according to embodiments 1-18, wherein the structural sleeve is sized for approximating the dimensions of the delivery chamber.

20. The silicone prosthesis delivery apparatus according to embodiments 1-19, wherein an outer surface of the structural sleeve provides markings or other indicia positioned and configured for assisting with the simultaneous cutting of the structural sleeve and delivery chamber to form the uniform trimmed opening.

21. The silicone prosthesis delivery apparatus according to embodiments 1-20, wherein the structural sleeve is constructed out of a flexible and relatively inelastic material.

22. The silicone prosthesis delivery apparatus according to embodiments 1-21, wherein the structural sleeve is constructed out of a film material having a lubricious additive dispersed therewithin.

23. The silicone prosthesis delivery apparatus according to embodiments 1-22, wherein the lubricious additive is a slip agent.

24. The silicone prosthesis delivery apparatus according to embodiments 1-23, wherein the structural sleeve further provides an anti-blocking additive.

25. The silicone prosthesis delivery apparatus according to embodiments 1-24, wherein the structural sleeve further provides a wax additive.

26. The silicone prosthesis delivery apparatus according to embodiments 1-25, wherein the structural sleeve provides a textured inner surface for enhancing lubricity of the structural sleeve.

27. The silicone prosthesis delivery apparatus according to embodiments 1-26, wherein an inner surface of the structural sleeve provides a lubricant.

28. The silicone prosthesis delivery apparatus according to embodiments 1-27, wherein an inner surface of the structural sleeve provides a hydrophilic coating.

29. The silicone prosthesis delivery apparatus according to embodiments 1-28, wherein the structural sleeve is transparent or semi-transparent.

30. The silicone prosthesis delivery apparatus according to embodiments 1-29, further comprising a sterile barrier packaging configured for storing and maintaining the sterilization of the delivery sleeve and prosthesis prior to use.

31. The silicone prosthesis delivery apparatus according to embodiments 1-30, wherein a tray of the barrier packaging contains a volume of an at least one fluid for hydrating the delivery sleeve and prosthesis.

32. A silicone prosthesis delivery apparatus for facilitating the insertion of a silicone prosthesis into a surgically developed pocket of a patient, the apparatus comprising: a substantially conical-shaped delivery chamber configured for receiving and subsequently expelling the prosthesis therefrom, the delivery chamber comprising a closed end and an opposing, relatively smaller diameter chamber opening configured for being selectively stretched to accommodate passage of the prosthesis into the delivery chamber and then released to retain the prosthesis within the delivery chamber; whereby, with the prosthesis positioned within the delivery chamber, the delivery chamber is capable of being manipulated to apply pressure to direct the prosthesis along a length of the delivery sleeve and toward the chamber opening, such that the prosthesis may be expelled from the delivery chamber through the chamber opening.

33. The silicone prosthesis delivery apparatus according to embodiment 32, wherein the delivery chamber is constructed out of a flexible material.

34. The silicone prosthesis delivery apparatus according to embodiments 32-33, wherein the deliver chamber is constructed out of a flexible material having elastic properties.

35. The silicone prosthesis delivery apparatus according to embodiments 32-34, wherein the delivery chamber is constructed out of a film material having a lubricious additive dispersed therewithin.

36. The silicone prosthesis delivery apparatus according to embodiments 32-35, wherein the lubricious additive is a slip agent.

37. The silicone prosthesis delivery apparatus according to embodiments 32-36, wherein the delivery chamber further provides an anti-blocking additive.

38. The silicone prosthesis delivery apparatus according to embodiments 32-37, wherein the delivery chamber further provides a wax additive.

39. The silicone prosthesis delivery apparatus according to embodiments 32-38, wherein the delivery chamber provides a textured inner surface for enhancing lubricity of the delivery chamber.

40. The silicone prosthesis delivery apparatus according to embodiments 32-39, wherein an inner surface of the delivery chamber provides a lubricant.

41. The silicone prosthesis delivery apparatus according to embodiments 32-40, wherein an inner surface of the delivery chamber provides a hydrophilic coating.

42. The silicone prosthesis delivery apparatus according to embodiments 32-41, wherein the delivery chamber is transparent or semi-transparent.

43. The silicone prosthesis delivery apparatus according to embodiments 32-42, wherein the diameter of the chamber opening is sized for preventing the prosthesis from unintentionally exiting the delivery chamber.

44. The silicone prosthesis delivery apparatus according to embodiments 32-43, wherein the delivery chamber is capable of being cut proximal the chamber opening, thereby allowing the chamber opening to be selectively enlarged to better accommodate the prosthesis as needed.

45. The silicone prosthesis delivery apparatus according to embodiments 32-44, wherein an outer surface of the delivery chamber provides markings or other indicia positioned and configured for assisting with the cutting of the delivery chamber to selectively enlarge the chamber opening.

46. The silicone prosthesis delivery apparatus according to embodiments 32-45, wherein the chamber opening provides a plurality of tabs extending outwardly therefrom, the tabs positioned and configured for facilitating the selective stretching of the chamber opening to accommodate passage of the prosthesis into the delivery chamber.

47. The silicone prosthesis delivery apparatus according to embodiments 32-46, wherein the delivery chamber is configured for being selectively cut at a location proximal the chamber opening, thereby separating the tabs from the delivery chamber and forming a trimmed opening.

48. The silicone prosthesis delivery apparatus according to embodiments 32-47, wherein an outer surface of the delivery chamber provides markings or other indicia positioned and configured for assisting with the cutting of the delivery sleeve to form the trimmed opening.

49. The silicone prosthesis delivery apparatus according to embodiments 32-48, further comprising: a substantially conical-shaped structural sleeve sized and configured for removably receiving the delivery chamber therewithin, thereby providing structural integrity to the delivery chamber; the structural sleeve having a first opening sized for receiving the delivery chamber therethrough, and an opposing, relatively smaller second opening sized for accommodating the chamber opening of the inserted delivery chamber; whereby, with the delivery chamber positioned within the structural sleeve, the delivery chamber and structural sleeve are configured for being simultaneously cut at a location proximal the second opening, thereby forming a uniform trimmed opening sized and configured for allowing the prosthesis to selectively pass therethrough when the prosthesis is expelled from the delivery chamber.

50. The silicone prosthesis delivery apparatus according to embodiments 32-49, wherein the structural sleeve is sized for approximating the dimensions of the delivery chamber.

51. The silicone prosthesis delivery apparatus according to embodiments 32-50, wherein an outer surface of the structural sleeve provides markings or other indicia positioned and configured for assisting with the simultaneous cutting of the structural sleeve and delivery chamber to form the uniform trimmed opening.

52. The silicone prosthesis delivery apparatus according to embodiments 32-51, wherein the structural sleeve is constructed out of a flexible and relatively inelastic material.

53. The silicone prosthesis delivery apparatus according to embodiments 32-52, wherein the structural sleeve is constructed out of a film material having a lubricious additive dispersed therewithin.

54. The silicone prosthesis delivery apparatus according to embodiments 32-53, wherein the lubricious additive is a slip agent.

55. The silicone prosthesis delivery apparatus according to embodiments 32-54, wherein the structural sleeve further provides an anti-blocking additive.

56. The silicone prosthesis delivery apparatus according to embodiments 32-55, wherein the structural sleeve further provides a wax additive.

57. The silicone prosthesis delivery apparatus according to embodiments 32-56, wherein the structural sleeve provides a textured inner surface for enhancing lubricity of the structural sleeve.

58. The silicone prosthesis delivery apparatus according to embodiments 32-57, wherein an inner surface of the structural sleeve provides a lubricant.

59. The silicone prosthesis delivery apparatus according to embodiments 32-58, wherein an inner surface of the structural sleeve provides a hydrophilic coating.

60. The silicone prosthesis delivery apparatus according to embodiments 32-59, wherein the structural sleeve is transparent or semi-transparent.

61. The silicone prosthesis delivery apparatus according to embodiments 32-60, further comprising a sterile barrier packaging configured for storing and maintaining the sterilization of the delivery chamber and prosthesis prior to use.

62. The silicone prosthesis delivery apparatus according to embodiments 32-61, wherein a tray of the barrier packaging contains a volume of an at least one fluid for hydrating the delivery chamber and prosthesis.

63. A method for preparing the silicone prosthesis delivery apparatus of embodiment 1, the method comprising the steps of: inserting a prosthesis through the entry opening of the transfer channel of the delivery sleeve, such that the prosthesis is positioned within the transfer channel; manipulating the transfer channel to apply pressure to direct the prosthesis along a length of the delivery sleeve and toward the delivery chamber, thereby urging the prosthesis through the passage and into the delivery chamber; separating the delivery chamber from the transfer channel, thereby forming the trimmed opening sized for preventing the prosthesis from unintentionally exiting the delivery chamber; and positioning the delivery sleeve within a sterile barrier packaging.

64. The method according to embodiment 63, further comprising the step of placing a volume of an at least one fluid into a tray of the barrier packaging for hydrating the delivery sleeve and prosthesis.

65. The method according to embodiments 63-64, wherein the step of separating the delivery chamber from the transfer channel further comprises the step of cutting the delivery chamber at a location proximal the passage, thereby separating the delivery chamber from the transfer channel and forming the trimmed opening.

66. The method according to embodiments 63-65, further comprising the steps of: inserting the delivery chamber into a substantially conical-shaped structural sleeve configured for providing structural integrity to the delivery chamber, the structural sleeve having a first opening sized for receiving the delivery chamber therethrough, and an opposing, relatively smaller second opening sized for accommodating the trimmed opening of the inserted delivery chamber; and with the delivery chamber positioned within the structural sleeve, simultaneously cutting the structural sleeve and delivery chamber at a location proximal the second opening, thereby forming a uniform trimmed opening sized and configured for allowing the prosthesis to selectively pass therethrough when the prosthesis is expelled from the delivery chamber.

In closing, regarding the exemplary embodiments of the present invention as shown and described herein, it will be appreciated that a silicone prosthesis delivery apparatus and configured for facilitating the insertion of a silicone prosthesis into a surgically developed pocket of a patient is disclosed. Because the principles of the invention may be practiced in a number of configurations beyond those shown and described, it is to be understood that the invention is not in any way limited by the exemplary embodiments, but is generally directed to a silicone prosthesis delivery apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112 (f) will begin with the words “means for,” but use of the term “for” in any other context is not intended to invoke treatment under 35 U.S.C. § 112 (f). Accordingly, Applicant reserves the right to pursue additional claims after filing this application, in either this application or in a continuing application.

It should be understood that the methods and the order in which the respective elements of each method are performed are purely exemplary. Depending on the implementation, they may be performed in any order or in parallel, unless indicated otherwise in the present disclosure.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicant and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A silicone prosthesis delivery apparatus for facilitating the insertion of a silicone prosthesis into a surgically developed pocket of a patient, the apparatus comprising:
a substantially hourglass-shaped delivery sleeve configured for receiving and subsequently expelling the prosthesis therefrom, the delivery sleeve comprising:
a substantially conical-shaped transfer channel and a substantially conical-shaped delivery chamber, the transfer channel and delivery chamber opposingly positioned and joined with one another so as to form a relatively smaller diameter passage therebetween;
a free end of the transfer channel providing an entry opening configured for allowing the prosthesis to selectively pass therethrough when the prosthesis is inserted into the delivery sleeve; and
a free end of the delivery chamber providing a closed end;
whereby, with the prosthesis positioned within the transfer channel, the transfer channel is capable of being manipulated to apply pressure to direct the prosthesis along a length of the delivery sleeve and toward the delivery chamber, such that the prosthesis may be urged through the passage and into the delivery chamber;
whereby, with the prosthesis subsequently positioned within the delivery chamber, the delivery chamber is configured for being selectively separated from the transfer channel at a location proximal the passage, thereby forming a trimmed opening at a proximal end of the delivery chamber, opposite the closed end, the trimmed opening sized and configured for allowing the prosthesis to selectively pass therethrough when the prosthesis is expelled from the delivery chamber; and
whereby, with the prosthesis positioned within the delivery chamber, the delivery chamber is capable of being manipulated to apply pressure to direct the prosthesis along a length of the delivery sleeve and toward the trimmed opening, such that the prosthesis may be expelled from the delivery chamber through the trimmed opening.

2. The silicone prosthesis delivery apparatus of claim 1, wherein the delivery sleeve is constructed out of a flexible material.

3. The silicone prosthesis delivery apparatus of claim 1, wherein the diameter of the passage is sized for preventing the prosthesis from unintentionally exiting the delivery chamber.

4. The silicone prosthesis delivery apparatus of claim 1, wherein the delivery chamber is configured for being selectively cut at a location proximal the passage, thereby separating the delivery chamber from the transfer channel.

5. The silicone prosthesis delivery apparatus of claim 1, further comprising:
a substantially conical-shaped structural sleeve sized and configured for removably receiving the delivery chamber therewithin after the delivery chamber has been separated from transfer channel, thereby providing structural integrity to the delivery chamber;
the structural sleeve having a first opening sized for receiving the delivery chamber therethrough, and an opposing, relatively smaller second opening sized for accommodating the trimmed opening of the inserted delivery chamber;
whereby, with the delivery chamber positioned within the structural sleeve, the delivery chamber and structural sleeve are configured for being simultaneously cut at a location proximal the second opening, thereby forming a uniform trimmed opening sized and configured for allowing the prosthesis to selectively pass therethrough when the prosthesis is expelled from the delivery chamber.

6. The silicone prosthesis delivery apparatus of claim 5, wherein the structural sleeve is sized for approximating the dimensions of the delivery chamber.

7. The silicone prosthesis delivery apparatus of claim 5, wherein the structural sleeve is constructed out of a flexible and relatively inelastic material.

8. The silicone prosthesis delivery apparatus of claim 1, further comprising a sterile barrier packaging configured for storing and maintaining the sterilization of the delivery sleeve and prosthesis prior to use.

9. A silicone prosthesis delivery apparatus for facilitating the insertion of a silicone prosthesis into a surgically developed pocket of a patient, the apparatus comprising:
a substantially conical-shaped delivery chamber configured for receiving and subsequently expelling the prosthesis therefrom, the delivery chamber comprising a closed end and an opposing, relatively smaller diameter chamber opening configured for being selectively stretched to accommodate passage of the prosthesis into the delivery chamber and then released to retain the prosthesis within the delivery chamber;
whereby, with the prosthesis positioned within the delivery chamber, the delivery chamber is capable of being manipulated to apply pressure to direct the prosthesis along a length of the delivery sleeve and toward the chamber opening, such that the prosthesis may be expelled from the delivery chamber through the chamber opening.

10. The silicone prosthesis delivery apparatus of claim 9, wherein the delivery chamber is constructed out of a flexible material.

11. The silicone prosthesis delivery apparatus of claim 9, wherein the diameter of the chamber opening is sized for preventing the prosthesis from unintentionally exiting the delivery chamber.

12. The silicone prosthesis delivery apparatus of claim 9, wherein the chamber opening provides a plurality of tabs extending outwardly therefrom, the tabs positioned and configured for facilitating the selective stretching of the chamber opening to accommodate passage of the prosthesis into the delivery chamber.

13. The silicone prosthesis delivery apparatus of claim 12, wherein the delivery chamber is configured for being selectively cut at a location proximal the chamber opening, thereby separating the tabs from the delivery chamber and forming a trimmed opening.

14. The silicone prosthesis delivery apparatus of claim 9, further comprising:
a substantially conical-shaped structural sleeve sized and configured for removably receiving the delivery chamber therewithin, thereby providing structural integrity to the delivery chamber; and
the structural sleeve having a first opening sized for receiving the delivery chamber therethrough, and an opposing, relatively smaller second opening sized for accommodating the chamber opening of the inserted delivery chamber;
whereby, with the delivery chamber positioned within the structural sleeve, the delivery chamber and structural sleeve are configured for being simultaneously cut at a location proximal the second opening, thereby forming a uniform trimmed opening sized and configured for allowing the prosthesis to selectively pass therethrough when the prosthesis is expelled from the delivery chamber.

15. The silicone prosthesis delivery apparatus of claim 14, wherein the structural sleeve is sized for approximating the dimensions of the delivery chamber.

16. The silicone prosthesis delivery apparatus of claim 14, wherein the structural sleeve is constructed out of a flexible and relatively inelastic material.

17. The silicone prosthesis delivery apparatus of claim 9, further comprising a sterile barrier packaging configured for storing and maintaining the sterilization of the delivery chamber and prosthesis prior to use.

18. A method for preparing the silicone prosthesis delivery apparatus of claim 1, the method comprising the steps of:
inserting a prosthesis through the entry opening of the transfer channel of the delivery sleeve, such that the prosthesis is positioned within the transfer channel;
manipulating the transfer channel to apply pressure to direct the prosthesis along a length of the delivery sleeve and toward the delivery chamber, thereby urging the prosthesis through the passage and into the delivery chamber;
separating the delivery chamber from the transfer channel, thereby forming the trimmed opening sized for preventing the prosthesis from unintentionally exiting the delivery chamber; and
positioning the delivery sleeve within a sterile barrier packaging.

19. The method of claim 18, wherein the step of separating the delivery chamber from the transfer channel further comprises the step of cutting the delivery chamber at a location proximal the passage, thereby separating the delivery chamber from the transfer channel and forming the trimmed opening.

20. The method of claim 18, further comprising the steps of:
inserting the delivery chamber into a substantially conical-shaped structural sleeve configured for providing structural integrity to the delivery chamber, the structural sleeve having a first opening sized for receiving the delivery chamber therethrough, and an opposing, relatively smaller second opening sized for accommodating the trimmed opening of the inserted delivery chamber; and
with the delivery chamber positioned within the structural sleeve, simultaneously cutting the structural sleeve and delivery chamber at a location proximal the second opening, thereby forming a uniform trimmed opening sized and configured for allowing the prosthesis to selectively pass therethrough when the prosthesis is expelled from the delivery chamber.
